(19) United States
(12) Patent Application Publication
(10) Pub. No.: US 2022/0389362 A1
(43) Pub. Date: Dec. 8, 2022

(54) CELL CULTURE APPARATUS

(71) Applicant: AMOGREENTECH CO., LTD., Gimpo-si (KR)

(72) Inventors: Hee Sung PARK, Gimpo-si (KR); In Yong SEO, Gimpo-si (KR); Seoung Hoon LEE, Gimpo-si (KR); Seon Ho JANG, Gimpo-si (KR); Su Yeon LEE, Gimpo-si (KR); Ji Young KIM, Gimpo-si (KR); Hyo Jung LEE, Gimpo-si (KR)

(21) Appl. No.: 17/754,995

(22) PCT Filed: Oct. 12, 2020
(86) PCT No.: PCT/KR2020/013857

§ 371 Date: Apr. 18, 2022

(30) Foreign Application Priority Data
Oct. 17, 2019 (KR) 10-2019-0129252

(51) Int. Cl. C12M 1/00; C12M 1/12; C12M 1/02; C12M 1/34

(52) U.S. Cl. CPC C12M 29/18; C12M 23/20; C12M 25/02; C12M 25/06; C12M 41/18; C12M 41/34

(57) ABSTRACT

A cell culture apparatus is provided. A cell culture apparatus according to one exemplary embodiment of the present invention comprises: a cell culture part comprising an accommodation space in which a plurality of supports for cell culture are disposed; a medium supply part for storing a predetermined amount of medium to be supplied to the cell culture part, and maintaining a predetermined carbon dioxide concentration of the medium stored therein by using carbon dioxide introduced from the outside through a gas supply port; and a pump, which interconnects the cell culture part and the medium supply part so as to circulate, in the cell culture part, a medium stored in the medium supply part.

### TECHNICAL FIELD

The present invention relates to a cell culture device.

### BACKGROUND ART

Cell culture is a method in which a tissue slice is removed from an individual of a multicellular organism and nourished to culture and proliferate cells in the tissue slice in a vessel.

In the field of biotechnology which has been rapidly developing since the 1980s, animal cell culture technology has played an important role in relation to the industrialization of biopharmaceuticals, and the importance of animal cell mass culture technology began to emerge since the mid-1980s.

Animal cells derived from human or animal tissues may be cultured by suspension in a culture medium or adhesion to a carrier. Mostly, blood cell-derived cells (including hematopoietic stem cells) are suspension cells, and cells derived from tissues such as skin, liver, or lungs and embryonic stem cells or mesenchymal stem cells, etc. are adherent cells. Suspension cells can proliferate alone in a state in which the cells are suspended in a culture medium, but adherent cells can proliferate only when adhered to a surface of a support body.

Accordingly, since suspension cells are advantageous for maintaining the highest cell density per unit volume during scale-up, the development of mass culture methods has mainly been made for suspension cells, and the development of devices for mass culture of adherent cells is insufficient.

### DISCLOSURE

### Technical Problem

The present invention is directed to providing a cell culture device which can be reduced in size and modularized and which is capable of mass culture of adherent cells through a single process.

### Technical Solution

The present invention provides a cell culture device including: a cell culture part which includes an accommodation space in which a plurality of support bodies for cell culture are disposed, a culture medium supply part which is configured to store a predetermined amount of culture medium for supply to the cell culture part and use carbon dioxide entering from the outside through a gas supply port to keep a carbon dioxide concentration of the culture medium stored therein constant, and a pump which is configured to interconnect the cell culture part and the culture medium supply part to allow the culture medium stored in the culture medium supply part to circulate through the cell culture part.

For example, the support body may include a motif-coated, plate-shaped nanofiber membrane. Specifically, the support body may include the motif-coated, plate-shaped nanofiber membrane and a support member which is attached to one surface of the nanofiber membrane via an adhesive layer to support the nanofiber membrane.

As another example, the support body may be a plasma-treated, plate-shaped film member.

Also, the cell culture part may include a culture housing formed in the shape of a vessel having an accommodation space, the plurality of support bodies which are disposed in multiple stages at predetermined intervals from each other in the accommodation space for cell culture and which are provided in the shape of a plate having a predetermined area, and a culture medium inlet and a culture medium outlet which are provided in the culture housing to allow the culture medium circulated through the pump to enter and exit.

Also, the culture medium supply part may include a culture medium housing which has an inner space formed to store the culture medium in a predetermined amount, a recovery port which is provided in the culture medium housing to recover the culture medium of the cell culture part to the inner space, a discharge port which is provided in the culture medium housing to supply the culture medium of the inner space to the cell culture part, and the gas supply port which is provided in the culture medium housing to allow carbon dioxide supplied from the outside to enter, wherein the inner space may be divided into a first space in which the culture medium is stored and a second space which is formed above the first space and filled with a gas including carbon dioxide, and the gas supply port may be provided in the culture medium housing to communicate with the second space.

Here, the culture medium supply part may include a filter member which is disposed in the second space to be spaced a predetermined distance apart from the culture medium filled in the inner space. In such a case, the filter member may be a nanofiber membrane or a gas-permeable film member.

Meanwhile, the culture medium supply part may further include a gas sensor configured to detect a carbon dioxide concentration of the second space, the culture medium supply part may further include a circulation fan configured to cause carbon dioxide entering through the gas supply port to circulate in the second space, and the cell culture device may further include a constant-temperature maintenance means configured to keep temperature of the cell culture part constant.

In such a case, the gas supply port may be a solenoid valve, and the constant-temperature maintenance means may be a heating jacket.

### Advantageous Effects

According to the present invention, since mass cell culture is possible through a single process and the consumption of culture medium can be reduced, cost reduction can be achieved.

Also, according to the present invention, there is an advantage in that size reduction of equipment is possible due to not requiring a separate incubator for keeping temperature and carbon dioxide concentration constant.

### DESCRIPTION OF DRAWINGS

FIG. 1 is a schematic diagram illustrating a cell culture device according to one embodiment of the present invention.

FIG. 2 is a view illustrating an exploded state of major components of FIG. 1.

FIG. 3 is a view illustrating a cell culture part which is applicable to the cell culture device according to one embodiment of the present invention.

FIG. 4 is an exploded view of FIG. 3.

FIG. 5 is a cross-sectional view taken along line A-A of FIG. 3.

FIG. 6 is a cross-sectional view taken along line B-B of FIG. 3.

FIG. 7 is a view illustrating a dispersion plate which is applicable to the cell culture part according to one embodiment of the present invention.

FIG. 8 is a view illustrating a case in which a support body, which is applicable to the cell culture device according to one embodiment of the present invention, includes a nanofiber membrane.

FIG. 9 is a view schematically illustrating a culture medium supply part which is applicable to the cell culture device according to one embodiment of the present invention.

FIG. 10 is a coupled cross-sectional view taken along line C-C of FIG. 9.

FIG. 11 is a view illustrating another form of culture medium housing that is applicable to FIG. 9.

FIG. 12 is a plan view of a state in which a portion of the culture medium housing is cut from FIG. 11.

### MODES OF THE INVENTION

Hereinafter, embodiments of the present invention will be described in detail with reference to the accompanying drawings to allow those of ordinary skill in the art to which the present invention pertains to easily carry out the present invention. The present invention may be implemented in various different forms and is not limited to the embodiments described herein. In the drawings, parts unrelated to the description have been omitted for clear description of the present invention, and like reference numerals are assigned to like or similar components throughout.

In a cell culture device 100 according to one embodiment of the present invention, in a state in which cells to be cultured are adhered to a plurality of support bodies 116, a culture medium whose pH level is kept constant is circulated to be supplied to the plurality of support bodies 116, and thus culture of the cells adhered to each of the support bodies 116 may be facilitated by nutrients supplied from the culture medium.

Also, the cell culture device 100 according to one embodiment of the present invention may culture cells using the plurality of support bodies 116 while being implemented in the form of a module with a reduced size, and thus a large number of cells can be cultured through a single process while the overall size of the cell culture device 100 is reduced.

Accordingly, the cell culture device 100 according to one embodiment of the present invention can, while securing mobility and work convenience, reduce the size of the overall equipment and reduce production costs.

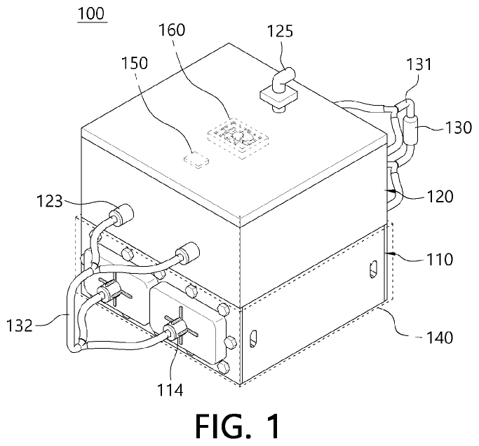

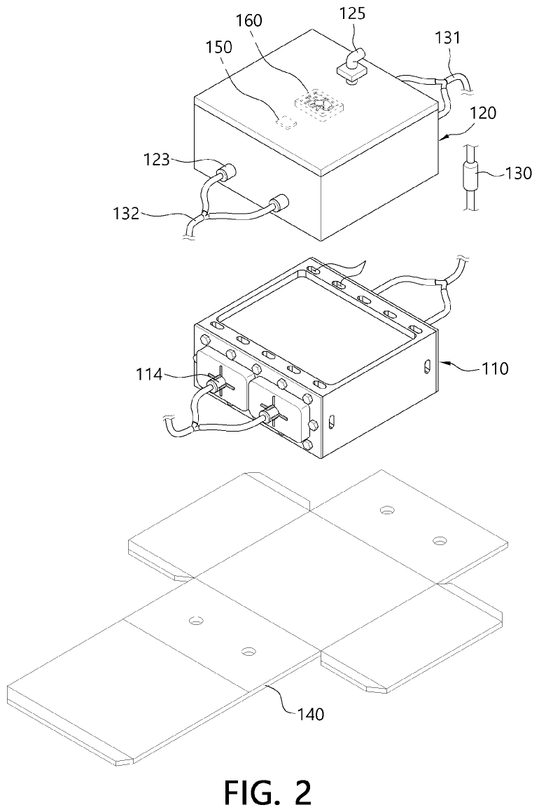

To this end, as illustrated in FIGS. 1 and 2, the cell culture device 100 according to one embodiment of the present invention includes a cell culture part 110, a culture medium supply part 120, and a pump 130.

The cell culture part 110 may have the plurality of support bodies 116 for cell culture mounted therein and may provide a space in which the cells adhered to the plurality of support bodies 116 are cultured.

Also, the cell culture part 110 may be connected to the culture medium supply part 120 via the pump 130, and by circulation of a culture medium stored in the culture medium supply part 120, a culture medium necessary for cell culture may be supplied to the cell culture part 110.

That is, cells to be cultured may be adhered to the plurality of support bodies 116, and the cells adhered to the support bodies 116 may be cultured by receiving nutrients through the culture medium supplied from the culture medium supply part 120.

Here, the plurality of support bodies 116 may be provided in the shape of a plate having a predetermined area and may be stacked in multiple stages in a state of being spaced apart from each other at predetermined intervals inside the cell culture part 110.

In this way, in the cell culture device 100 according to one embodiment of the present invention, by increasing a degree of integration of the plurality of support bodies 116 mounted in the cell culture part 110, mass cell culture can be possible through a single process.

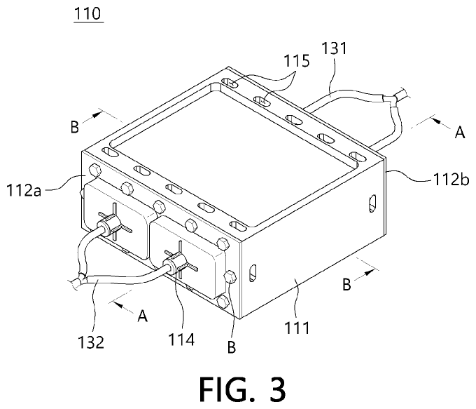

To this end, as illustrated in FIGS. 3 to 6, the cell culture part 110 may include a culture housing 111, the plurality of support bodies 116, a culture medium inlet 113, and a culture medium outlet 114.

The culture housing 111 may accommodate the plurality of support bodies 116 and the culture medium. To this end, the culture housing 111 may be formed in the shape of a vessel having an accommodation space S1.

For example, as illustrated in FIG. 3, the culture housing 111 may be formed in the shape of a vessel having the accommodation space S1 whose front surface and rear surface are open.

In such a case, a first cap part 112a in which at least one culture medium inlet 113 is formed and a second cap part 112b in which at least one culture medium outlet 114 is formed may be respectively coupled to the open front surface and rear surface of the culture housing 111.

Also, the culture medium inlet 113 may be connected to the culture medium supply part 120 via the pump 130, and the culture medium outlet 114 may be connected to the culture medium supply part 120 via a connecting line 132. Here, the pump 130 may be disposed on a connecting line 131 configured to interconnect the culture medium inlet 113 of the cell culture part 110 and a discharge port 124 of the culture medium supply part 120 but may also be disposed on the connecting line 132 configured to interconnect the culture medium outlet 114 of the cell culture part 110 and a recovery port 123 of the culture medium supply part 120.

In this way, the culture medium stored in the culture medium supply part 120 may, through the pump 130, circulate inside the accommodation space S1 of the cell culture part 110 and circulate inside the culture medium supply part 120, and the culture medium supplied from the culture medium supply part 120 may fill the accommodation space S1 through the culture medium inlet 113.

Accordingly, the plurality of support bodies 116 disposed in the accommodation space S1 may be submerged in the culture medium supplied into the accommodation space S1, and the cells adhered to each of the support bodies 116 may receive nutrients necessary for cell culture from the culture medium.

Here, in a case in which the front surface and rear surface of the culture housing 111 are closed, the culture medium inlet 113 and the culture medium outlet 114 may be directly formed in the front surface and rear surface, respectively, of the culture housing 111.

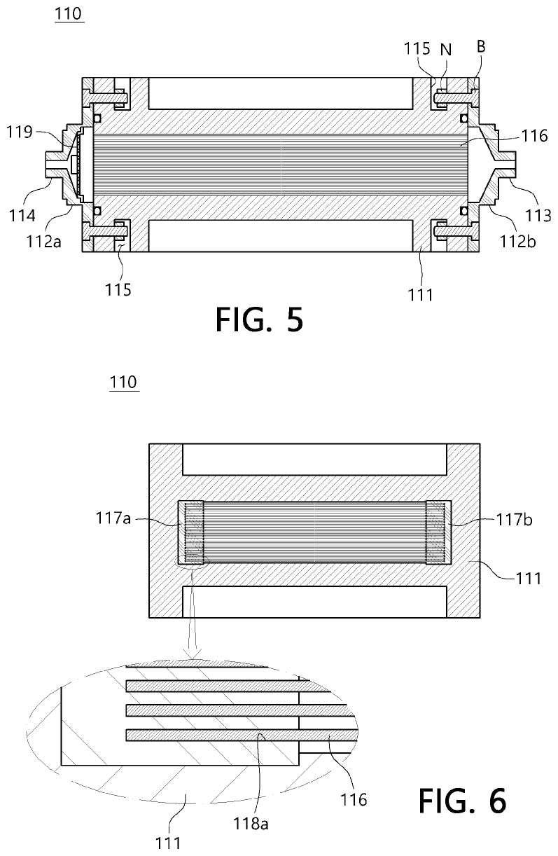

Also, an accommodation hole 115 recessed inward as illustrated in FIG. 5 may be formed in one side of the culture housing 111. In a case in which the first cap part 112a and the second cap part 112b are fastened to the culture housing 111 through a bolt member B, an end portion of the bolt member B may protrude toward the accommodation hole 115, and a nut member N for fixing the bolt member B may be accommodated in the accommodation hole 115. Accordingly, by fastening the bolt member B and the nut member N using the accommodation hole 115, a worker may couple the first cap part 112a and the second cap part 112b to the culture housing 111.

Cells to be cultured may be adhered to at least one surface of the plurality of support bodies 116.

As described above, the support bodies 116 may be provided in the shape of a plate having a predetermined area to culture a large number of cells through one culture and may be stacked in multiple stages in the accommodation space S1 of the culture housing 111.

Here, various known materials used in cell culture may be used as a material of each of the support bodies 116 without limitations as long as the support bodies 116 can be implemented in the plate shape and cells can be easily adhered to the support bodies 116.

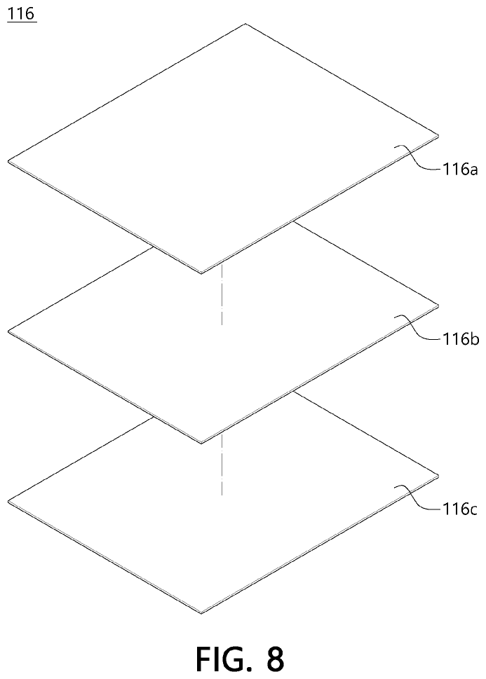

For example, the support body 116 may include a nanofiber membrane 116a in which nanofibers are formed in a three-dimensional network structure by electrospinning. In such a case, as illustrated in FIG. 8, the support body 116 may have a three-layer structure which includes the nanofiber membrane 116a and further includes a support member 116c attached to one surface of the nanofiber membrane 116a via an adhesive layer 116b.

Here, the support member 116c may be a plate-shaped film member and may support the one surface of the nanofiber membrane 116a. In this way, even when the nanofiber membrane 116a, while flexible, is formed in the shape of a plate, the nanofiber membrane 116a may be supported by the support member 116c, and thus bending or sagging of the nanofiber membrane 116a may be prevented. Accordingly, since the support bodies 116 disposed in the accommodation space S1 of the culture housing 111 may remain unfolded, cell culture may be facilitated.

However, types of the support bodies 116 used in the present invention are not limited to the above. Each support body may be configured as a plate-shaped film member having a predetermined area, and various known materials used in cell culture may be used as a material of each support body as long as each support body can be implemented in the shape of a plate and cells can be easily adhered to each support body.

Here, surfaces of the support bodies 116 may be modified to facilitate adhesion of cells to be cultured. For example, in a case in which the support body 116 includes the nanofiber membrane 116a, the nanofiber membrane 116a may be a membrane in which motif is applied to a surface of nanofibers. Also, in a case in which the support body is provided as a plate-shaped film member, the film member may be a plasma-treated film member.

Accordingly, cells to be cultured may be easily adhered to surfaces of the support bodies 116, and the cells to be cultured may be, in a state of being adhered to the surfaces of the support bodies 116, cultured by nutrients supplied from the culture medium.

Here, the cell culture device 100 according to one embodiment of the present invention may include a constant-temperature maintenance means 140 configured to maintain the temperature of the cell culture part 110 to a temperature suitable for cell culture.

The constant-temperature maintenance means 140 may be disposed to surround peripheral surfaces of the cell culture part 110. For example, the constant-temperature maintenance means 140 may be disposed to surround peripheral surfaces of the culture housing 111. Accordingly, a temperature of the accommodation space S1 of the culture housing 111 may be maintained to a temperature suitable for cell culture by the constant-temperature maintenance means 140, and thus cell culture may be facilitated.

As a non-limiting example, the constant-temperature maintenance means 140 may be a known heating jacket but is not limited thereto, and any other heating means can be applied without limitations as long as the heating means can provide heat to maintain a certain temperature.

In this way, the cell culture device 100 according to one embodiment of the present invention may maintain the temperature of the cell culture part 110 to a suitable temperature by the constant-temperature maintenance means 140, and thus a separate incubator for keeping the temperature constant during cell culture may not be necessary.

Accordingly, the cell culture device 100 according to one embodiment of the present invention may be configured in units of modules through a configurations of the culture medium supply part 120 which will be described below, and thus each configuration may be modularized.

Thus, the cell culture device 100 according to one embodiment of the present invention may allow the entire system for cell culture to be constructed through simple connections and may overcome a limitation of requiring construction of a separate space for cell culture, such as an incubator.

Meanwhile, in the cell culture device 100 according to one embodiment of the present invention, the plurality of support bodies 116 disposed in multiple stages in the accommodation space S1 may be disposed in the accommodation space S1 in a state of being spaced apart from each other at predetermined intervals to, while increasing the degree of integration, facilitate supply of nutrients from the culture medium to the cells adhered to each of the support bodies 116.

To this end, a spacer may be disposed in the accommodation space S1 of the culture housing 111 to allow the plurality of support bodies 116 to be accommodated in the state of being spaced apart from each other at predetermined intervals.

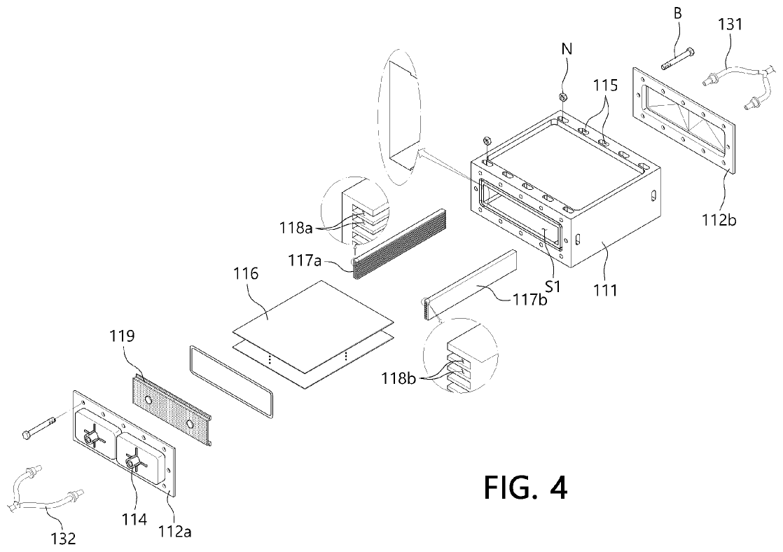

For example, as illustrated in FIGS. 4 and 6, the spacer may include two guide members 117a and 117b inserted into the accommodation space S1 so that one surface of one guide member and one surface of the other guide member face each other. Here, the two guide members 117a and 117b may be a first guide member 117a and a second guide member 117b which are disposed at the left side in FIG. 3.

In such a case, a plurality of slot grooves 118a and 118b formed to be recessed in a height direction may be formed in opposing surfaces of the first guide member 117a and the second guide member 117b that face each other, and the first guide member 117a and the second guide member 117b may be disposed so that surfaces in which the slot grooves 118a and 118b are not formed, which are opposite to the surfaces in which the slot grooves 118a and 118b are formed, come into contact with two inner surfaces that face each other among inner surfaces of the culture housing 111.

In this way, the first guide member 117a and the second guide member 117b may be disposed in the accommodation space S1 so that the surfaces in which the slot grooves 144a and 144b are formed face each other.

Accordingly, when each of the support bodies 116 is inserted into the slot grooves 118a and 118b in a state in which the first guide member 117a and the second guide member 117b are inserted into the accommodation space S1, each of both side ends of the support bodies 116 may be inserted into one of the slot grooves 118a formed in the first guide member 117a and the slot grooves 118b formed in the second guide member 117b.

Thus, due to both side ends of the support bodies 116 being constrained by the slot grooves 118a and 118b, the support bodies 116 may be disposed in a horizontal state in the accommodation space S1, and two support bodies 116 that are adjacent to each other may remain spaced apart by as much as an interval between two slot grooves formed in the height direction.

In this way, both surfaces of the plurality of support bodies 116 disposed in multiple stages in the accommodation space S1 may easily come into contact with the culture medium filled in the accommodation space S1, and culture of the cells adhered to the support bodies 116 may be facilitated by nutrients supplied from the culture medium.

In this way, since the plurality of support bodies 116 may be mounted by sliding in the cell culture device 100 according to one embodiment of the present invention, convenience of assembly can be improved.

However, the total number of guide members constituting the spacer is not limited to the above, and the spacer may consist of three or more guide members according to the total number of support bodies 116 to be mounted. The number of guide members may be set to any number without limitations as long as the guide members form a pair with each other.

Further, the spacer may have any suitable shape other than the shape including the above-described guide member having the slot groove formed therein, as long as the spacer can maintain a state in which the plate-shaped support bodies are spaced apart from each other at predetermined intervals.

For example, the spacer may include a fastening bar (not illustrated) configured to simultaneously fasten the plurality of support bodies 116 and a ring-shaped ring member fitted to the fastening bar. In such a case, the ring member may be disposed between two support bodies 116. In this way, the plurality of support bodies 116 may remain spaced apart from each other by as much as a thickness of the ring member.

As another example, the spacer may be configured by combination of the guide member having the slot groove formed therein, the fastening bar, and the ring member.

Meanwhile, an inner surface of the first cap part 112a in which the culture medium inlet 113 is formed may be formed to be recessed inward with respect to the culture medium inlet 113.

That is, as illustrated in FIGS. 4 and 5, the inner surfaces of the cap parts 112a and 112b in which the culture medium inlet 113 is formed may have a concave shape in which a cross-sectional area gradually increases from an end portion of the culture medium inlet 113 in a direction in which the culture medium moves, and the end portion of the culture medium inlet 113 may form a central portion having the concave shape.

In this way, the culture medium entering the cell culture part 110 through the culture medium inlet 113 may easily enter the accommodation space S1.

Here, plate-shaped dispersion plates 119 and 219 having a predetermined area may be disposed between the culture medium inlet 113 and the support body 116 disposed in the accommodation space S1, and the dispersion plates 119 and 219 may be disposed to be spaced a predetermined distance apart from an end portion of the support body 116 mounted in the accommodation space S1.

The dispersion plates 119 and 219 may impede the culture medium, which enters the cell culture part 110 through the culture medium inlet 113, from immediately moving into the accommodation space S1.

That is, the culture medium entering the cell culture part 110 through the culture medium inlet 113 may collide with the dispersion plates 119 and 219 and be dispersed evenly. In this way, the culture medium evenly dispersed in the process of colliding with the dispersion plates 119 and 219 may, regardless of the positions of the plurality of support bodies 116 mounted in the accommodation space S1, move into all separation spaces formed between the support bodies 116 at one time, and thus supply of the culture medium toward each of the support bodies 116 may be facilitated.

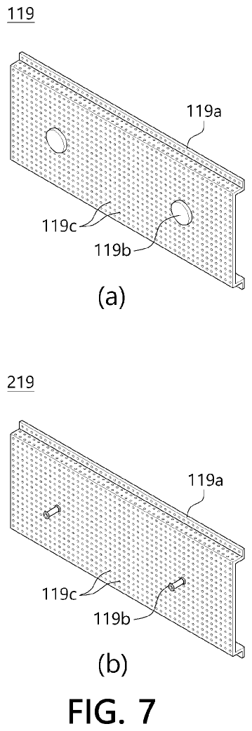

For example, as illustrated in FIG. 7, the dispersion plates 119 and 219 may have a form in which a plurality of through-holes 119c are formed to pass through a plate-shaped body 119a having a predetermined area, but the dispersion plates 119 and 219 are not limited thereto and may also be a mesh in which a plurality of through-holes are formed.

Here, an impeding means 119b configured to block the culture medium from immediately moving may be formed at positions on the dispersion plates 119 and 219 that correspond to the culture medium inlet 113.

For example, as illustrated in FIG. 7(a), the impeding means 119b of the dispersion plates 119 and 219 may be portions of the body 119a where the through-holes 119c are not formed so that the culture medium is not able to immediately pass therethrough.

Alternatively, as illustrated in FIG. 7(b), the impeding means 119b of the dispersion plates 119 and 219 may be protruding portions formed to protrude from the body 119a of the dispersion plate toward the culture medium inlet 113. In such a case, one end portion of the protruding portion may be disposed at a close distance from the end portion of the culture medium inlet 113 and cause the culture medium entering from the culture medium inlet 113 to immediately collide with the end portion of the protruding portion. In this way, the culture medium may be dispersed more effectively.

The culture medium containing nutrients necessary for cell culture may be stored in a predetermined amount in the culture medium supply part 120, and the culture medium stored in the culture medium supply part 120 may be circulated to the cell culture part 110 through the pump 130.

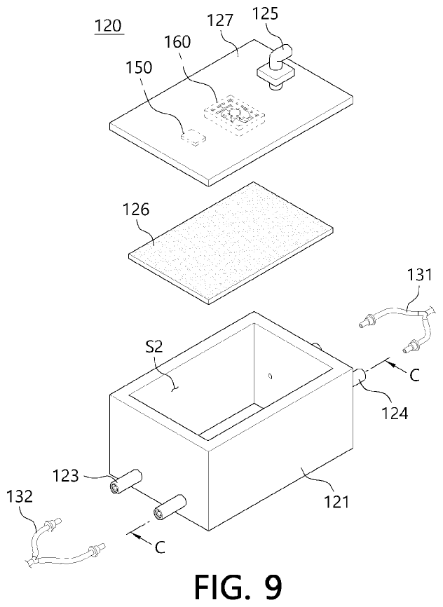

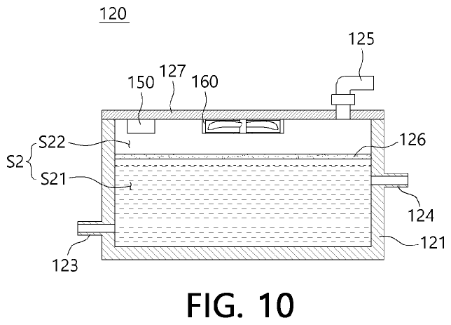

To this end, as illustrated in FIGS. 9 to 12, the culture medium supply part 120 may include culture housings 121 and 221 in the shape of a vessel having an inner space S2 for storing the culture medium in a predetermined amount, and in order to allow the culture medium stored in the inner space S2 to be recovered after being circulated and supplied to the cell culture part 110 during operation of the pump 130, the culture housings 121 and 221 may include the recovery port 123 and the discharge port 124 through which the culture medium enters or exits.

Here, the inner space S2 may be formed to have an open upper portion. In such a case, the culture housings 121 and 221 may further include a lid member 127 configured to cover the open upper portion of the inner space S2.

Also, the discharge port 124 may be connected to the culture medium inlet 113 of the cell culture part 110 via the pump 130 and the connecting line 131, and the recovery port 123 may be connected to the culture medium outlet 114 of the cell culture part 110 via the connecting line 132. In this way, the culture medium stored in the inner space S2 may be circulated by the operation of the pump 130, and the culture medium stored in the inner space S2 may be supplied to the cell culture part 110 and then be recovered to the culture medium supply part 120.

Further, regarding the culture medium filling the inner space S2, an appropriate amount of the culture medium may fill the inner space S2 with a predetermined water level instead of completely filling the inner space S2. That is, the inner space S2 may be divided into a first space S21 in which the culture medium is stored up to a predetermined height of the entire height and a second space S22 which is formed above the first space S21 and filled with a gas including carbon dioxide, and the first space S21 and the second space S22 may be changed according to the amount of storage of the culture medium filled in the inner space S2. In such a case, a boundary line that distinguishes the first space S21 and the second space S22 may be a water level of the culture medium filled in the inner space S2.

Accordingly, the gas present in the second space S22 may be dissolved in the culture medium filled in the first space S21, and the amount of carbon dioxide may be kept constant in the culture medium. Thus, the culture medium stored in the first space S21 may maintain a pH level suitable for cell culture and then may be supplied to the cell culture part 110.

Here, the culture medium supply part 120 may include a gas supply port 125 provided in the culture housings 121 and 221 to allow carbon dioxide to enter the second space S22 from the outside. The gas supply port 125 may be connected to a separate carbon dioxide supply means (not illustrated) and may be disposed in the culture housings 121 and 221 to communicate with the second space S22.

For example, the gas supply port 125 may be provided in the lid member 127 and may be a known solenoid valve but is not limited thereto. Any other known valve may be used without limitations as long as the valve is able to be opened/closed and can adjust a supply amount.

In this way, even when the amount of carbon dioxide dissolved in the culture medium decreases in the process in which the culture medium is recovered to the first space S21 through the recovery port 123 after moving to the cell culture part 110, the culture medium recovered to the first space S21 may receive carbon dioxide from the second space S22 while staying in the first space S21.

Thus, the culture medium circulated from the first space S21 to the cell culture part 110 may be resupplied to the cell culture part 110 after the pH level of the culture medium is changed to a pH level suitable for cell culture. In this way, cell culture may be facilitated.

Thus, even when the culture medium is repeatedly circulated in the cell culture part 110 and the culture medium supply part 120 through the pump 130 during cell culture, the cells adhered to the support bodies 116 may continuously receive the culture medium which is in a state suitable for culturing. In this way, culturing of the cells may be facilitated.

Here, the culture medium supply part 120 may further include a gas sensor 150 configured to detect a concentration of carbon dioxide present in the second space S22 and may further include a circulation fan 160 configured to circulate the gas of the second space S22 so that carbon dioxide entering the second space S22 through the gas supply port 125 is rapidly dispersed in the second space S22.

The gas sensor 150 and the circulation fan 160 may be disposed in the culture housings 121 and 221 to be placed at the second space S22 side. For example, the gas sensor 150 and the circulation fan 160 may be disposed to be placed on an inner surface of the lid member 127.

Accordingly, in the cell culture device 100 according to one embodiment of the present invention, the amount of carbon dioxide supplied into the second space S22 through the gas supply port 125 may be controlled on the basis of information detected by the gas sensor 150. Thus, the amount of carbon dioxide dissolved in the culture medium stored in the first space S21 may be changed to reach a certain pH level, and the pH level may be maintained by controlling the amount of carbon dioxide entering the second space S22 through the gas supply port 125.

Here, the operation of the gas sensor 150, the circulation fan 160, the gas supply port 125, etc. may be controlled by a separate controller (not illustrated).

Also, a filter member 126 may be disposed at the second space S22 side to be spaced a predetermined distance apart from the culture medium filled in the first space S21. The filter member 126 may be made of a material that blocks entry of foreign matter but allows passage of carbon dioxide. For example, the filter member 126 may be a nanofiber membrane or a gas-permeable film member.

In this way, the gas present in the second space S22 may pass through the filter member 126 and move toward the culture medium stored in the first space S21, and the gas in a state in which foreign matter is filtered therefrom may be supplied toward the culture medium.

Accordingly, the amount of dissolved carbon dioxide may be kept constant in the culture medium filled in the first space S21, and contamination of the culture medium due to foreign matter included in the gas may be prevented.

Meanwhile, the culture medium supply part 120 may be formed so that the discharge port 124 is placed at a relatively lower position than the recovery port 123. That is, the discharge port 124 may be formed at a position relatively closer to bottom surfaces of the culture housings 121 and 221, and the recovery port 123 may be formed at a position relatively farther from the bottom surfaces of the culture housings 121 and 221.

In this way, the culture medium entering the first space S21 from the cell culture part 110 through the recover port 123 may move to the cell culture part 110 through the discharge port 124 formed at a relatively low position.

Thus, even when the culture medium recovered from the cell culture part 110 to the first space S21 through the recovery port 123 includes air bubbles, the air bubbles included in the culture medium may move upward due to buoyancy in the process in which the culture medium moves toward the discharge port 124 formed at a relatively lower position than the recover port 123.

In this way, the culture medium supplied toward the cell culture part 110 through the discharge port 124 may not include air bubbles. Accordingly, the cells adhered to the support bodies 116 may easily receive nutrients from the culture medium without interference of air bubbles, and thus culturing of the cells may be facilitated.

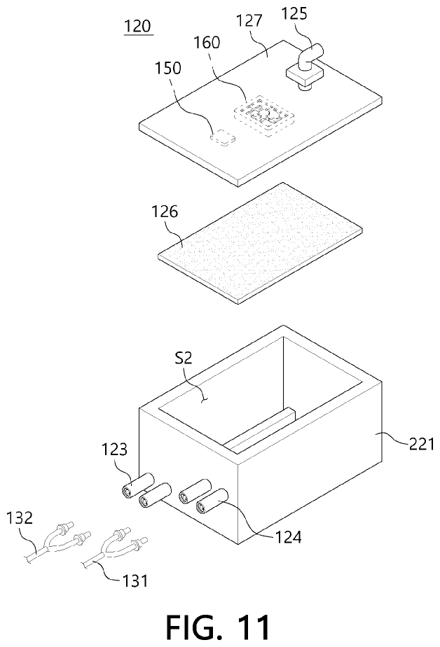

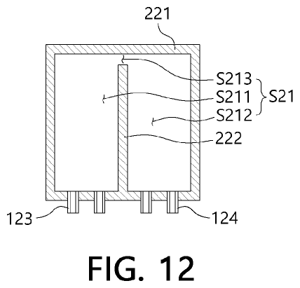

Meanwhile, in the culture medium supply part 120 that is applicable to the cell culture device 100 according to one embodiment of the present invention, the first space S21 in which the culture medium is stored may be divided into at least two spaces as illustrated in FIGS. 11 and 12.

To this end, the culture medium supply part 120 may include at least one partition 222 formed to protrude from the bottom surface of the culture housing 221, and the first space S21 formed in the culture housing 221 may be divided into a culture medium recovery space S211 and a culture medium supply space S212 via the partition 222.

Here, the partition 222 may be formed to protrude from the bottom surface of the culture housing 221 so that one end portion is connected to an inner side surface of the culture housing 221 and the other end portion is spaced a predetermined distance apart from another inner side surface that faces the inner side surface of the culture housing 221 to which the one end portion is connected.

Accordingly, the culture medium recovery space S211 and the culture medium supply space S212 may communicate with each other through a communication path S213 formed between an end portion of the partition 222 and an inner side surface of the culture housing 221 that faces the end portion.

In such a case, the recovery port 123 may be formed at a position that allows communication with the culture medium recovery space S211, and the discharge port 124 may be formed at a position that allows communication with the culture medium supply space S212.

Further, as described above, the recovery port 123 may be formed to be placed at a relatively higher position than the discharge port 124. That is, the discharge port 124 may be formed at a position relatively closer to the bottom surface of the culture housing 221, and the recovery port 123 may be formed at a position relatively farther from the bottom surface of the culture housing 221.

In this way, the culture medium entering the culture medium supply part 120 may be discharged to the outside through the discharge port 124 after moving a relatively longer distance as compared to the culture housing 121 which has the form described above. That is, the culture medium discharged from the cell culture part 110 may enter the culture medium recovery space S211 and then move toward the culture medium supply space S212 via the communication path S213, and in this way, a moving distance along which the culture medium is discharged to the outside through the discharge port 124 may be increased.

Thus, sufficient carbon dioxide may be supplied to the culture medium in the process in which the culture medium moves from the recovery port 123 to the discharge port 124, and air bubbles included in the culture medium may float due to buoyancy and be completely separated from the culture medium in the process in which the culture medium moves from the recovery port 123 to the discharge port 124.

Accordingly, the culture medium supplied to the cell culture part 110 through the discharge port 124 may be in a best state in which impurities such as air bubbles have been removed from the culture medium. Accordingly, culture of the cells adhered to the support bodies 116 may be further facilitated.

In this way, by interconnecting the culture medium supply part 120, the pump 130, and the cell culture part 110 through the connecting lines 131 and 132 and configuring the culture medium to circulate in the culture medium supply part 120 and the cell culture part 110 by the operation of the pump 130, a closed circulation system may be simply implemented in the cell culture device 100 according to one embodiment of the present invention.

Further, in the cell culture device 100 according to one embodiment of the present invention, the pH level of the culture medium circulated from the culture medium supply part 120 to the cell culture part 110 through the pump 130 is maintained at a certain level in the culture medium supply part 120 using carbon dioxide supplied through the gas supply port 125, and thus the use amount of culture medium necessary for cell culture may be minimized.

In addition, in the cell culture device 100 according to one embodiment of the present invention, a circulation method in which the culture medium is circulated is employed, carbon dioxide for keeping the pH level of the circulated culture medium constant is supplied through the gas supply port 125 of the culture medium supply part 120, and constant-temperature maintenance for keeping the temperature of the cell culture part 110 constant during cell culture may be implemented using a constant-temperature maintenance means such as a heating jacket coupled to the cell culture part 110.

Thus, in the cell culture device 100 according to one embodiment of the present invention, the amount of carbon dioxide dissolved in the culture medium may be kept constant, and an environment for cell culture may be constructed without a separate closed space such as an incubator for maintaining an appropriate temperature necessary for cell culture.

In this way, the cell culture device 100 according to one embodiment of the present invention may be implemented in the form of a module with a reduced size and can minimize limitations in terms of space for cell culture.

That is, in the cell culture device 100 according to one embodiment of the present invention, the entire system for cell culture may be simply implemented when a separate carbon dioxide supply means is connected to the gas supply port 125 in a state in which the culture medium supply part 120 is stacked on the cell culture part 110 and the pump 130 is installed on the connecting lines 131 and 132 configured to interconnect the culture medium supply part 120 and the cell culture part 110. In this way, the entire system for cell culture can be reduced in size and modularized, and mass culture of cells can be stably cultured.

Embodiments of the present invention have been described above, but the spirit of the present invention is not limited to the embodiments proposed herein. Those of ordinary skill in the art who understand the spirit of the present invention may easily propose other embodiments by addition, alteration, omission, etc. of components within the scope of the same spirit, but such embodiments also belong to the scope of the spirit of the present invention.

1. A cell culture device comprising:
a cell culture part which includes an accommodation space in which a plurality of support bodies for cell culture are disposed;
a culture medium supply part which is configured to store a predetermined amount of culture medium for supply to the cell culture part and use carbon dioxide entering from the outside through a gas supply port to keep a carbon dioxide concentration of the culture medium stored therein constant; and
a pump which is configured to interconnect the cell culture part and the culture medium supply part to allow the culture medium stored in the culture medium supply part to circulate through the cell culture part.

2. The cell culture device of claim 1, wherein the support body includes a motif-coated, plate-shaped nanofiber membrane.

3. The cell culture device of claim 2, wherein the support body includes the motif-coated, plate-shaped nanofiber membrane and a support member which is attached to one surface of the nanofiber membrane via an adhesive layer to support the nanofiber membrane.

4. The cell culture device of claim 1, wherein the support body is a plasma-treated, plate-shaped film member.

5. The cell culture device of claim 1, wherein the cell culture part includes a culture housing formed in the shape of a vessel having an accommodation space, the plurality of support bodies which are disposed in multiple stages at predetermined intervals from each other in the accommodation space for cell culture and which are provided in the shape of a plate having a predetermined area, and a culture medium inlet and a culture medium outlet which are provided in the culture housing to allow the culture medium circulated through the pump to enter and exit.

6. The cell culture device of claim 5, wherein:
the cell culture part further includes two guide members inserted into the accommodation space so that one surface of one guide member and one surface of the other guide member face each other; and
the two guide members include a plurality of slot grooves formed to be recessed in a longitudinal direction so that end sides of the support bodies are able to be fitted to the surfaces facing each other.

7. The cell culture device of claim 1, wherein:
the culture medium supply part includes a culture medium housing which has an inner space formed to store the culture medium in a predetermined amount, a recovery port which is provided in the culture medium housing to recover the culture medium of the cell culture part to the inner space, a discharge port which is provided in the culture medium housing to supply the culture medium of the inner space to the cell culture part, and the gas supply port which is provided in the culture medium housing to allow carbon dioxide supplied from the outside to enter, and
the inner space is divided into a first space in which the culture medium is stored and a second space which is formed above the first space and filled with a gas including carbon dioxide, and the gas supply port is provided in the culture medium housing to communicate with the second space.

8. The cell culture device of claim 7, wherein the culture medium supply part includes a filter member which is disposed in the second space to be spaced a predetermined distance apart from the culture medium filled in the inner space.

9. The cell culture device of claim 8, wherein the filter member is a nanofiber membrane or a gas-permeable film member.

10. The cell culture device of claim 7, wherein the culture medium supply part further includes a gas sensor configured to detect a carbon dioxide concentration of the second space.

11. The cell culture device of claim 7, wherein the culture medium supply part further includes a circulation fan configured to cause carbon dioxide entering through the gas supply port to circulate in the second space.

12. The cell culture device of claim 1, wherein the gas supply port is a solenoid valve.

13. The cell culture device of claim 1, further comprising a constant-temperature maintenance means configured to keep a temperature of the cell culture part constant.

14. The cell culture device of claim 13, wherein the constant-temperature maintenance means is disposed to surround peripheral surfaces of the cell culture part.

15. The cell culture device of claim 13, wherein the constant-temperature maintenance means is a heating jacket.
